(19) United States
(12) Patent Application Publication
(10) Pub. No.: US 2019/0321005 A1
(43) Pub. Date: Oct. 24, 2019

(54) SUBJECT INFORMATION ACQUISITION APPARATUS, SUBJECT INFORMATION PROCESSING METHOD, AND STORAGE MEDIUM USING PROBE TO RECEIVE ACOUSTIC WAVE

(71) Applicant: CANON KABUSHIKI KAISHA, Tokyo (JP)

(72) Inventors: Shoya Sasaki, Yokohama-shi (JP); Kenichi Nagae, Yokohama-shi (JP)

(21) Appl. No.: 16/383,376

(22) Filed: Apr. 12, 2019

(30) Foreign Application Priority Data
Apr. 18, 2018 (JP) 2018-080092

(51) Int. Cl. A61B 8/08; A61B 5/00; A61B 8/00

(52) U.S. Cl. CPC A61B 8/5207; A61B 8/42; A61B 5/0059; A61B 5/0095

(57) ABSTRACT

A subject information acquisition apparatus includes a light emission unit, a probe, and image generation unit, and a display control unit. The light emission unit is configured to emit light to a subject. The probe is configured to receive an acoustic wave generated from the subject to which the light is emitted, thereby generating a signal. The image generation unit is configured to generate a plurality of frame images based on signals acquired at a plurality of respective relative positions of the probe to the subject and resulting from acoustic waves from the subject. The display control unit is configured to selectively display images of an area common to consecutive frames in the plurality of frame images at a display unit.

### BACKGROUND OF THE INVENTION

### Field of the Invention

One disclosed aspect of the embodiments relates to a subject information acquisition apparatus, a subject information processing method, and a storage medium.

### Description of the Related Art

Photoacoustic imaging (PAI) is a method for acquiring optical property information within a living body. In photoacoustic imaging, based on an acoustic wave generated by the photoacoustic effect (hereinafter also referred to as a “photoacoustic wave”) from a subject irradiated with pulsed light, it is possible to acquire optical property information in the subject and generate an image based on the acquired optical property information.

Japanese Patent Application Laid-Open No. 2012-179348 discusses an acoustic wave acquisition apparatus that changes the relative position between a detector for receiving a photoacoustic wave and a subject and receives photoacoustic waves from the subject at a plurality of relative positions. Further, Japanese Patent Application Laid-Open No. 2012-179348 discusses a technique for displaying an image in real time while acquiring photoacoustic waves by the detector scanning the subject.

First, an issue is described that can occur in a conventional acoustic wave acquisition apparatus.

As illustrated in FIG. 7A, a probe moves along a circular trajectory and receives photoacoustic waves from a subject when the probe is located at three measurement positions Pos1, Pos2, and Pos3. That is, when the probe is located at the measurement positions Pos1, Pos2, and Pos3, light that induces a photoacoustic wave is emitted to the subject.

FIGS. 7B, 7C, and 7D illustrate images Im1, Im2, and Im3 generated based on the photoacoustic waves received at the measurement positions Pos1, Pos2, and Pos3, respectively, by the probe. A dotted line indicates a display area DA on a display unit. The area where the probe can receive a photoacoustic wave with excellent sensitivity is determined based on the placement of acoustic wave detection elements included in the probe. Thus, the positions on the display area DA of the images Im1, Im2, and Im3 generated based on the photoacoustic waves received at the measurement positions Pos1, Pos2, and Pos3, respectively, also fluctuate in conjunction with the measurement positions Pos1, Pos2, and Pos3. As discussed in Japanese Patent Application Laid-Open No. 2012-179348, if a moving image is displayed, the positions where the images Im1, Im2, and Im3 are formed are different from one another, and thus, the range where the image is displayed on the display area DA fluctuates with respect to each frame. Thus, in an area not common to consecutive frames, the image may be displayed or may not be displayed with respect to each frame. The phenomenon is recognized as flicker in the moving image by an observer and causes the observer's stress.

### SUMMARY OF THE INVENTION

According to an aspect of the embodiments, a subject information acquisition apparatus includes a light emission unit, a probe, and image generation unit, and a display control unit. The light emission unit is configured to emit light to a subject. The probe is configured to receive an acoustic wave generated from the subject to which the light is emitted, thereby generating a signal. The image generation unit is configured to generate a plurality of frame images based on signals acquired at a plurality of respective relative positions of the probe to the subject and resulting from acoustic waves from the subject. The display control unit is configured to selectively display images of an area common to consecutive frames in the plurality of frame images at a display unit.

Further features of the disclosure will become apparent from the following description of exemplary embodiments with reference to the attached drawings.

### BRIEF DESCRIPTION OF THE DRAWINGS

FIG. 1 is a block diagram illustrating a configuration of a subject information acquisition apparatus according to an exemplary embodiment.

FIG. 2 is a diagram illustrating an example of a configuration of a probe according to the exemplary embodiment.

FIG. 3 is a diagram illustrating a processing flow according to the exemplary embodiment.

FIG. 4 is a diagram illustrating a state of a movement of the probe according to the exemplary embodiment.

FIG. 5 is a diagram illustrating a display screen according to the exemplary embodiment.

FIG. 6 is a diagram illustrating timings of light emissions and acquisition and generation of signals according to the exemplary embodiment.

FIGS. 7A to 7D are diagrams illustrating an issue that can occur in a conventional acoustic wave acquisition apparatus.

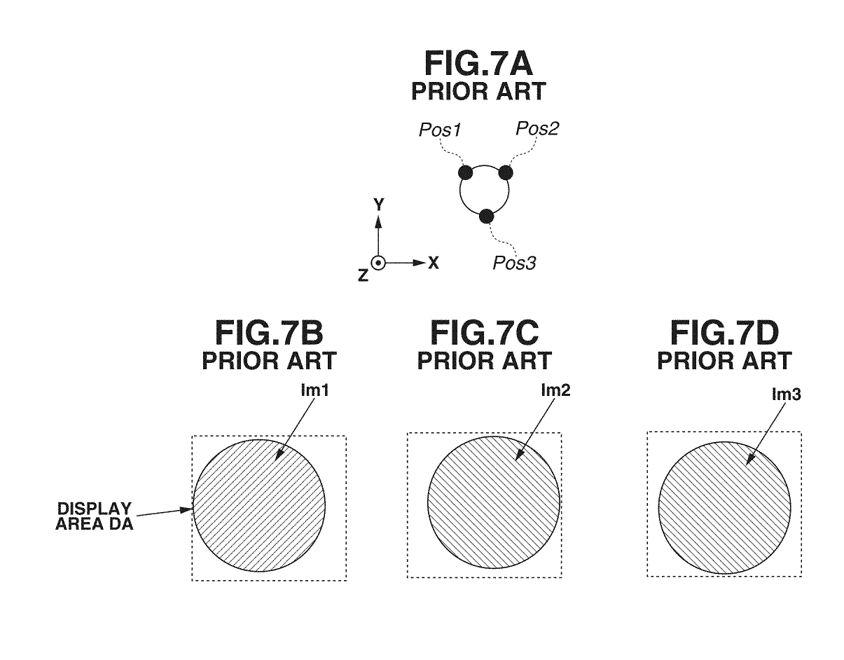

### DESCRIPTION OF THE EMBODIMENTS

In the following exemplary embodiments of the disclosure, images of an area common to consecutive frames are selectively displayed, thereby reducing flicker in a moving image.

With reference to the drawings, exemplary embodiments will be described below. However, the dimensions, the materials, the shapes, and the relative arrangement of the following components should be appropriately changed based on the configuration of an apparatus to which the disclosure is applied, or various conditions. Thus, the scope of the disclosure is not limited to the following description.

Photoacoustic image data obtained by an apparatus described below reflects the amount of absorption and the absorption rate of light energy. The photoacoustic image data is image data representing the spatial distribution of subject information about at least one of the generation sound pressure (the initial sound pressure) of a photoacoustic wave, the light absorption energy density, and the light absorption coefficient, and the concentration of a substance forming subject tissue. The concentration of the substance is, for example, the oxygen saturation distribution, the total hemoglobin concentration, or the oxidation-reduction hemoglobin concentration. The photoacoustic image data may be image data representing a two-dimensional spatial distribution, or may be image data representing a three-dimensional spatial distribution.

A first exemplary embodiment is described. In the present exemplary embodiment, the area in a subject where pieces of photoacoustic image data are generated from photoacoustic signals acquired by changing the relative position between the subject and a probe and the display area of photoacoustic images are the same. Display is updated using as the display area the area where the pieces of photoacoustic image data are generated, and the pieces of photoacoustic image data are displayed as a moving image, whereby it is possible to continuously observe a certain observation range.

The configuration of a subject information acquisition apparatus and an information processing method according to the present exemplary embodiment will be described below.

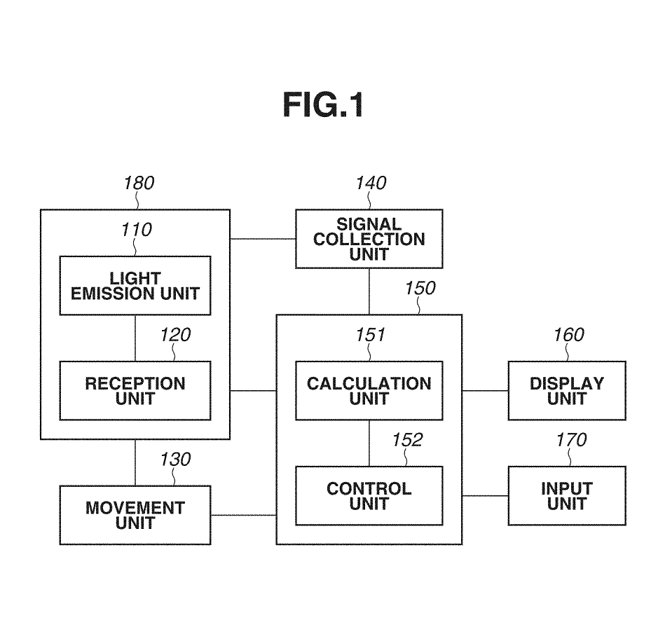

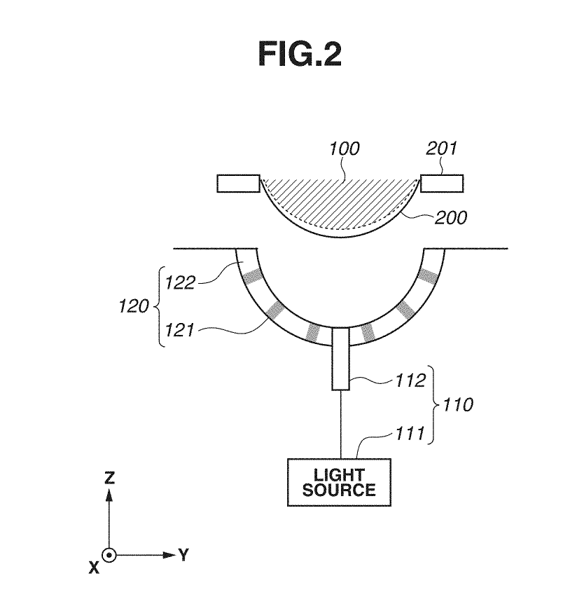

FIG. 1 is a block diagram illustrating the configuration of the subject information acquisition apparatus according to the present exemplary embodiment. FIG. 2 is a diagram illustrating an example of the configuration of a probe 180 according to the present exemplary embodiment. The subject information acquisition apparatus according to the present exemplary embodiment includes a movement unit 130, a signal collection unit 140, a computer 150, a display unit 160, an input unit 170, and a probe 180. The probe 180 includes a light emission unit 110 and a reception unit 120 and hereinafter will also be referred to as a “probe”. The movement unit 130 drives the light emission unit 110 and the reception unit 120 to perform mechanical scanning so as to change the relative positions of the light emission unit 110 and the reception unit 120 to a subject 100. The light emission unit 110 emits light to the subject 100, and an acoustic wave is generated in the subject 100. The acoustic wave generated by the photoacoustic effect due to the light is also referred to as a “photoacoustic wave”. The reception unit 120 receives the photoacoustic wave, thereby outputting an electric signal (a photoacoustic signal) as an analog signal.

The signal collection unit 140 converts the analog signal output from the reception unit 120 into a digital signal and outputs the digital signal to the computer 150. The computer 150 stores the digital signal output from the signal collection unit 140, as signal data resulting from a photoacoustic wave.

The computer 150 functions as a control unit for controlling the subject information acquisition apparatus and also as an image generation unit for generating image data. The computer 150 performs signal processing on a stored digital signal, thereby generating image data representing a photoacoustic image. Further, the computer 150 performs image processing on the obtained image data and then outputs the image data to the display unit 160. The display unit 160 displays the photoacoustic image based on the image data. A doctor or a technologist as a user of the apparatus can make a diagnosis by confirming the photoacoustic image displayed at the display unit 160. Based on a saving instruction from the user or the computer 150, the image displayed at the display unit 160 is saved in a memory or a storage unit in the computer 150 or a data management system connected to a modality via a network. As discussed in the following, the computer 150 may be a processor, a programmable device, or a central processing unit (CPU) that may execute a program or instructions stored in a storage device such as memory to perform operations described in the following.

Further, the computer 150 also controls the driving of the components included in the subject information acquisition apparatus. Furthermore, the display unit 160 may display a graphical user interface (GUI) in addition to the image generated by the computer 150. The input unit 170 is configured to enable the user to input information. Using the input unit 170, the user can perform the operation of starting or ending measurements, or the operation of giving an instruction to save a created image.

The details of the components of the subject information acquisition apparatus according to the present exemplary embodiment will be described below.

### (Light Emission Unit 110)

The light emission unit 110 includes a light source 111 that emits light, and an optical system 112 that guides the light emitted from the light source 111 to the subject 100. Examples of the light include pulse light having a square wave or a triangle wave.

The pulse width of the light emitted from the light source 111 may be a pulse width of 1 ns or more and 100 ns or less. Further, the wavelength of the light may be a wavelength in the range of about 400 nm to 1600 nm. In a case where blood vessels are imaged at high resolution, a wavelength of which light is largely absorbed in the blood vessels (400 nm or more and 700 nm or less) may be used. In a case where a deep part of a living body is imaged, light of a wavelength that is typically less absorbed in background tissue (water or fat) of the living body (700 nm or more and 1100 nm or less) may be used.

As the light source 111, a laser or a light-emitting diode can be used. Further, when measurements are made using light of a plurality of wavelengths, a light source capable of changing its wavelength may be used. In a case where light with a plurality of wavelengths is emitted to the subject 100, it is also possible to prepare a plurality of light sources for generating light of different wavelengths and alternately emit light from the light sources. Also in a case where a plurality of light sources is used, the plurality of light sources is collectively referred to as a “light source”. As the laser, various lasers such as a solid-state laser, a gas laser, a dye laser, and a semiconductor laser can be used. For example, as the light source 111, a pulse laser such as a neodymium-doped yttrium aluminum garnet (Nd:YAG) laser or an alexandrite laser may be used. Alternatively, as the light source 111, a titanium-sapphire (Ti:sa) laser or an optical parametric oscillator (OPO) laser, which uses Nd:YAG laser light as excitation light, may be used. Yet alternatively, as the light source 111, a flash lamp or a light-emitting diode may be used. Yet alternatively, as the light source 111, a microwave source may be used.

As the optical system 112, optical elements such as a lens, a mirror, and an optical fiber can be used. In a case where the breast is the subject 100, then to emit pulse light by expanding the beam diameter of the pulse light, a light exit portion of the optical system 112 may be composed of a diffusion plate for diffusing light. On the other hand, in a photoacoustic microscope, to increase resolution, the light exit portion of the optical system 112 may be composed of a lens and emit a beam by focusing the beam.

The light emission unit 110 may not include the optical system 112, and the light source 111 may directly emit light to the subject 100.

### (Reception Unit 120)

The reception unit 120 includes transducers 121 that each receive an acoustic wave, thereby outputting a signal, typically an electric signal, and a supporting member 122 that supports the transducers 121. Not only can each transducer 121 receive an acoustic wave, but the transducer 121 can also be used as a transmission unit for transmitting an acoustic wave. A transducer as a reception unit and a transducer as a transmission unit may be a single (common) transducer, or may be separately provided.

The transducer 121 can be configured using a piezoelectric ceramic material typified by lead zirconate titanate (PZT) or a piezoelectric polymer membrane material typified by polyvinylidene difluoride (PVDF). Alternatively, an element other than an element using a piezoelectric element may be used. For example, a transducer using a capacitance transducer (a capacitive micromachined ultrasonic transducer (CMUT)) can be used. Any transducer may be employed so long as the transducer can output a signal according to the reception of an acoustic wave. Further, a signal obtained by the transducer 121 is a time-resolved signal. That is, the amplitude of the signal obtained by the transducer 121 represents a value based on sound pressure (e.g., a value proportional to the sound pressure) received by the transducer 121 at each time.

Frequency components included in a photoacoustic wave are typically from 100 KHz to 100 MHz. Thus, as the transducer 121, a transducer capable of detecting these frequencies can be employed.

To define the relative positions among the plurality of transducers 121, the supporting member 122 may be composed of a metal material having high mechanical strength. To make a large amount of emitted light incident on the subject 100, a mirror surface may be provided or processing for scattering light may be performed on the surface on the subject 100 side of the supporting member 122. In the present exemplary embodiment, the supporting member 122 has a hemispherical shell shape and is configured to support the plurality of transducers 121 on the hemispherical shell. In this case, the directional axes of the transducers 121 placed in the supporting member 122 concentrate near the curvature center of the hemisphere. Then, when an image is formed using signals output from the plurality of transducers 121, the image quality near the curvature center is high. The area is referred to as a “high-resolution area”. The high-resolution area refers to an area where half or more of the receiving sensitivity at the position of the maximum receiving sensitivity defined based on the placement of the plurality of transducers 121 is obtained. In the configuration of the present exemplary embodiment, the curvature center of the hemisphere is an area where the maximum receiving sensitivity is achieved, and the high-resolution area is a spherical area isotropically spreading from the center of the hemisphere. It is desirable to control the movement unit 130 and the light emission unit 110 so that light is emitted to the subject 100 while the movement unit 130 moves the probe 180 by a distance less than or equal to the size of the high-resolution area. In this manner, in acquired pieces of data, it is possible to cause the areas of the high-resolution areas to overlap each other. The supporting member 122 may have any configuration so long as the supporting member 122 can support the transducers 121. In the supporting member 122, the plurality of transducers 121 may be arranged on a flat surface or a curved surface termed a 1D array, a 1.5D array, a 1.75D array, or a 2D array. The plurality of transducers 121 corresponds to a plurality of acoustic wave detection units. Also in a 1D array and a 1.5D array, a high-resolution area determined based on the placement of transducers exists.

Further, the supporting member 122 may function as a container for storing an acoustic matching material. That is, the supporting member 122 may be a container for placing an acoustic matching material between the transducers 121 and the subject 100.

Furthermore, the reception unit 120 may include an amplifier for amplifying a time-series analog signal output from each transducer 121. Further, the reception unit 120 may include an analog-to-digital (A/D) converter for converting the time-series analog signal output from the transducer 121 into a time-series digital signal. That is, a configuration may be employed in which the reception unit 120 includes the signal collection unit 140.

The space between the reception unit 120 and the subject 100 is filled with a medium through which a photoacoustic wave can propagate. It is desirable to employ as the medium a material through which an acoustic wave can propagate, and of which the acoustic properties match at an interface with the subject 100 or the transducers 121, and which has high transmittance of a photoacoustic wave. For example, as the medium, water or ultrasonic gel can be employed.

FIG. 2 illustrates a cross-sectional view of the probe 180. The probe 180 according to the present exemplary embodiment includes the reception unit 120, in which the plurality of transducers 121 is placed along the spherical surface of the hemispherical supporting member 122 that includes an opening. Further, the light exit portion of the optical system 112 is placed in a bottom portion in a z-axis direction of the supporting member 122.

In the present exemplary embodiment, as illustrated in FIG. 2, the subject 100 comes into contact with a retention member 200, whereby the shape of the subject 100 is retained.

The space between the reception unit 120 and the retention member 200 is filled with a medium through which a photoacoustic wave can propagate. It is desirable to employ as the medium a material through which a photoacoustic wave can propagate, and of which the acoustic properties match at an interface with the subject 100 or the transducers 121, and which has high transmittance of a photoacoustic wave. For example, as the medium, water or ultrasonic gel can be employed.

The retention member 200 as a retention unit is used to retain the shape of the subject 100 while the subject 100 is measured. The retention member 200 retains the subject 100 and thereby can restrain the movement of the subject 100 and maintain the position of the subject 100 within the retention member 200. As the material of the retention member 200, a resin material such as polycarbonate, polyethylene, or polyethylene terephthalate can be used.

The retention member 200 is attached to an attachment portion 201. The attachment portion 201 may be configured such that a plurality of types of retention members 200 can be replaced based on the size of the subject 100. For example, the attachment portion 201 may be configured such that the retention member 200 can be replaced with another retention member 200 having a different radius of curvature or a different curvature center. The attachment portion 201 can be placed in, for example, an opening portion provided in a bed. This enables an examinee to insert a part to be examined into the opening portion in a seated, prone, or supine position on the bed.

### (Movement Unit 130)

The movement unit 130 includes a component for changing the relative position between the subject 100 and the reception unit 120. The movement unit 130 includes a motor, such as a stepper motor, for generating a driving force, a driving mechanism for transmitting the driving force, and a position sensor for detecting position information regarding the reception unit 120. As the driving mechanism, a lead screw mechanism, a link mechanism, a gear mechanism, or a hydraulic mechanism can be used. Further, as the position sensor, a potentiometer using an encoder, a variable resistor, a linear scale, a magnetic sensor, an infrared sensor, or an ultrasonic sensor can be used.

The movement unit 130 may change the relative position between the subject 100 and the reception unit 120 not only in an XY direction (two-dimensionally), but also in one-dimensionally or three-dimensionally.

The movement unit 130 may fix the reception unit 120 and move the subject 100 so long as the movement unit 130 can change the relative position between the subject 100 and the reception unit 120. In a case where the subject 100 is moved, it is possible to employ a configuration in which the subject 100 is moved by moving the retention member 200 retaining the subject 100. Alternatively, both the subject 100 and the reception unit 120 may be moved.

The movement unit 130 may continuously change the relative position, or may change the relative position by a step-and-repeat process. The movement unit 130 may be an electric stage for changing the relative position on a programmed trajectory, or may be a manual stage.

Furthermore, in the present exemplary embodiment, the movement unit 130 simultaneously drives the light emission unit 110 and the reception unit 120, thereby performing scanning Alternatively, the movement unit 130 may drive only the light emission unit 110, or may drive only the reception unit 120. That is, although FIG. 2 illustrates a case where the light emission unit 110 is formed integrally with the supporting member 122, the light emission unit 110 may be provided independently of the supporting member 122.

### (Signal Collection Unit 140)

The signal collection unit 140 includes an amplifier for amplifying an electric signal, which is an analog signal output from each transducer 121, and an A/D converter for converting the analog signal output from the amplifier into a digital signal. The signal collection unit 140 may be composed of a field-programmable gate array (FPGA) chip. The digital signal output from the signal collection unit 140 is stored in the storage unit in the computer 150. The signal collection unit 140 is also termed a data acquisition system (DAS). In the specification, an electric signal is a concept including both an analog signal and a digital signal. A light detection sensor such as a photodiode may detect the emission of light from the light emission unit 110, and the signal collection unit 140 may start the above processing in synchronization with the detection result as a trigger. The light detection sensor may detect light coming out of the exit end of the optical system 112, or may detect light on an optical path from the light source 111 to the optical system 112. Further, the signal collection unit 140 may start the processing in synchronization with an instruction given using a freeze button as a trigger.

### (Computer 150)

The computer 150 includes a calculation unit 151 and a control unit 152. In the present exemplary embodiment, the computer 150 functions both as an image data generation unit and a display control unit. The functions of these components will be described in the description of a processing flow.

A unit having a calculation function as the calculation unit 151 can be composed of a processor such as a central processing unit (CPU) or a graphics processing unit (GPU), or an arithmetic circuit such as an FPGA chip. The unit may be composed of not only a single processor or a single arithmetic circuit, but also a plurality of processors or a plurality of arithmetic circuits. The calculation unit 151 may receive various parameters such as the sound speed of the subject 100 and the configuration of the retention member 200 from the input unit 170 and process a reception signal.

The control unit 152 is composed of an arithmetic element such as a CPU. The control unit 152 controls the operations of the components of the subject information acquisition apparatus. The control unit 152 may receive instruction signals based on various operations such as starting measurements from the input unit 170 and control the components of the subject information acquisition apparatus. Further, the control unit 152 reads a program code stored in the storage unit and executes the program to control the operations of the components of the subject information acquisition apparatus.

The calculation unit 151 and the control unit 152 may be achieved by common hardware, or specialized circuits to perform the operations

Further, as the storage unit, either a volatile memory, such as random access memory (RAM), or a non-volatile memory, such as Flash electrically erasable read only memory (EEROM), can be used so long as the purpose can be achieved.

### (Display Unit 160)

The display unit 160 is a display such as a liquid crystal display or an organic electroluminescent (EL) display. The display unit 160 may display a GUI for operating an image or the apparatus.

### (Input Unit 170)

As the input unit 170, an operation console that can be operated by the user and is composed of a mouse and a keyboard can be employed. Alternatively, the display unit 160 may be composed of a touch panel, and the display unit 160 may also be used as the input unit 170.

### (Subject 100)

Although not included in the subject information acquisition apparatus, the subject 100 will be described below. The subject information acquisition apparatus according to the present exemplary embodiment can be used to diagnose a malignant tumor or a blood vessel disease of a person or an animal, or to perform a follow-up of a chemical treatment. Thus, as the subject 100, a diagnosis target part such as a living body, e.g., the breast, each organ, a network of blood vessels, the head, the neck, the abdomen, or four limbs including fingers or toes of a human body or an animal, is assumed. For example, if a human body is a measurement target, oxyhemoglobin, deoxyhemoglobin, blood vessels including a large amount of oxyhemoglobin or deoxyhemoglobin, or new blood vessels formed near a tumor may be a target of radiographic contrasting, and light of a wavelength with a high absorption coefficient of hemoglobin may be emitted to the target. Alternatively, melanin, collagen, or a lipid included in the skin may be a target of a light absorber. Further, a pigment such as methylene blue (MB) or indocyanine green (ICG), gold microparticles, or an externally introduced substance obtained by accumulating or chemically modifying these materials may be a light absorber. Furthermore, a phantom simulating a living body may be the subject 100.

The components of the subject information acquisition apparatus may be configured as different devices, or may be configured as a single integrated device. Further, at least some of the components of the subject information acquisition apparatus may be configured as a single integrated device.

Apparatuses included in a system according to the present exemplary embodiment may be configured by different pieces of hardware, or all the apparatuses may be configured by a single piece of hardware. The functions of the system according to the present exemplary embodiment may be configured by any hardware.

### (Flow for Observing Subject Information in Moving Image)

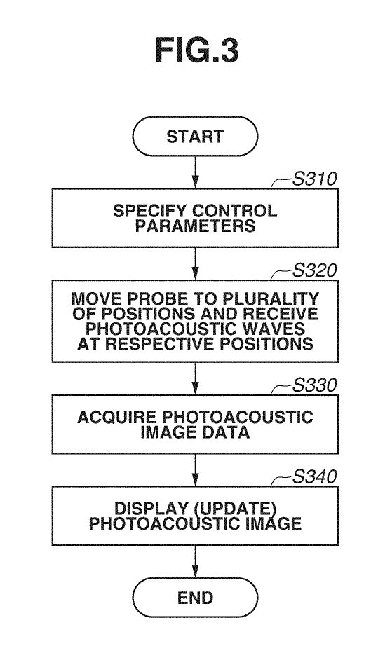

FIG. 3 illustrates the flow for observing subject information in a moving image.

### (Step 5310: Process of Specifying Control Parameters)

Using the input unit 170, the user specifies control parameters, such as emission conditions (the repetition frequency of light emission, the wavelength, and the intensity of light) of the light emission unit 110 and a measurement range (a region of interest (ROI)), that are necessary to acquire subject information. The computer 150 sets the control parameters determined based on instructions given by the user through the input unit 170. Further, the time in which a moving image of subject information is acquired may be set. At this time, the user may also be allowed to give instructions for the frame rate and the resolution of the moving image.

### (Step 5320: Process of Moving Probe to Plurality of Positions and Receiving Photoacoustic Waves at Respective Positions).

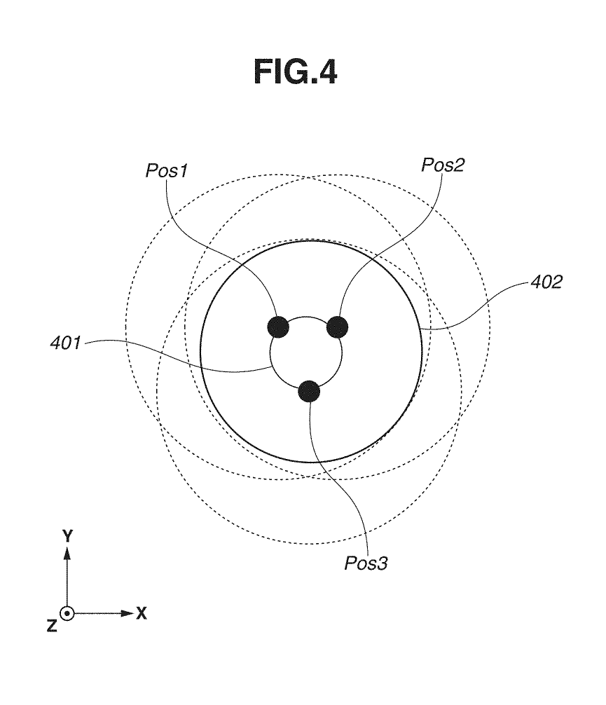

FIG. 4 is a diagram schematically illustrating the movement of the probe 180 in the present exemplary embodiment. FIG. 4 illustrates a diagram obtained by, in a case where the movement unit 130 moves the probe 180 in an xy-plane, projecting a trajectory 401 drawn by the center of the probe 180 onto the xy-plane. Particularly, in the configuration illustrated in FIG. 2, the trajectory 401 can also be said to be a trajectory drawn by the center of the light emission unit 110 or the center of the high-resolution area projected onto the xy-plane. In FIG. 4, positions Pos1, Pos2, and Pos3 represent the positions of the center of the probe 180 at timings Te1, Te2, and Te3, respectively, when light is emitted to the subject 100. Circles indicated by dotted lines centered at the points of the positions Pos1, Pos2, and Pos3 each schematically illustrate the expansion of an area on the subject 100 irradiated with the light or the high-resolution area at the position. In this case, for ease of description, a case is described where the area irradiated with the light and the high-resolution area coincide with each other. These areas, however, may not need to completely coincide with each other. For example, these areas may have such a relationship that one includes the other, or may have such a relationship that only parts of these areas overlap each other.

Based on the control parameters specified in step S310, the light emission unit 110 emits light to the subject 100 at the positions Pos1, Pos2, and Pos3 in FIG. 4. At this time, the light emission unit 110 transmits a synchronization signal to the signal collection unit 140 simultaneously with the transmission of pulse light. If receiving the synchronization signal transmitted from the light detection sensor having detected the emission of the light from the light emission unit 110, the signal collection unit 140 starts the operation of collecting a signal at each of the positions Pos1, Pos2, and Pos3 in FIG. 4. That is, the signal collection unit 140 amplifies and performs AD conversion on an analog electric signal resulting from an acoustic wave and output from the reception unit 120, thereby generating an amplified digital electric signal. Then, the signal collection unit 140 outputs the amplified digital electric signal to the computer 150. The computer 150 stores, together with the digital electric signal, position information obtained when the probe 180 receives the acoustic wave, in association with the digital electric signal.

### (Step S330: Process of Acquiring Photoacoustic Image Data)

Based on signal data output from the signal collection unit 140 in step S320, the calculation unit 151 of the computer 150 as an image acquisition unit generates photoacoustic image data.

The calculation unit 151 generates a plurality of pieces of photoacoustic image data V1 to V3 based on signal data obtained by emitting light once. Then, the calculation unit 151 combines the plurality of pieces of photoacoustic image data V1 to V3, thereby calculating combined photoacoustic image data V′1 in which an artifact is reduced. The pieces of photoacoustic image data V1 to V3 are pieces of photoacoustic image data generated based on acoustic waves received when the probe 180 is located at the respective positions Pos1, Pos2, and Pos3. In a case where a moving image is generated as an image of one frame every time light is emitted once, the pieces of photoacoustic image data V1 to V3 correspond to three consecutive frames.

A display area 402 illustrated in FIG. 4 represents the range of an area, in a spatial coordinate system including the subject 100 and the probe 180, for displaying subject information as a photoacoustic image at the display unit 160. The display area 402 may be an area representing a three-dimensional spatial distribution, or may be an area representing a two-dimensional spatial distribution. In a case where the display area 402 represents a three-dimensional spatial distribution, a two-dimensional spatial distribution based on the photoacoustic image of the display area 402, such as a maximum intensity projection (MIP) from a particular direction or a cross-sectional image based on a particular position, may be displayed at the display unit 160.

In the present exemplary embodiment, the display area 402 in FIG. 4 and the areas of the pieces of photoacoustic image data generated by the calculation unit 151 are the same. That is, for example, the high-resolution area when the probe 180 is located at the position Pos1 corresponds to the range of the circle indicated by the dotted line centered at the position Pos1 in FIG. 4. The calculation unit 151, however, generates photoacoustic data only regarding the range of the display area 402 indicated by a solid line. This can also be said to be the generation of photoacoustic image data by differentiating the spatial center of the high-resolution area and the spatial center of the area where the photoacoustic image data is generated. As described above, although the area corresponding to the high-resolution area is actually wider than the display area 402, the range where the photoacoustic image data is generated is narrower than the high-resolution area, whereby it is possible to reduce the processing load of the calculation unit 151. This is effective in a case where a moving image is displayed in real time during measurements.

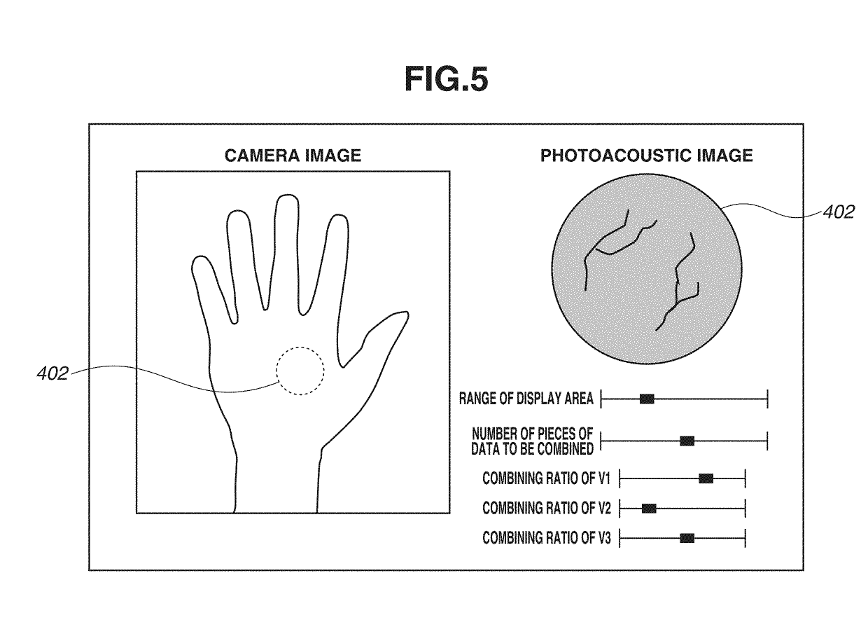

FIG. 5 illustrates an example of a display screen displayed at the display unit 160 according to the present exemplary embodiment. In a case where, as in FIG. 5, the system includes a camera as an image capturing unit for imaging the entirety or a part of the subject 100, the position and the range of the display area 402 relative to the subject 100 may be specified based on a camera image (hereinafter also referred to as an “optical image”). The user may be allowed to drag the display area 402 indicated on the camera image, using a mouse, thereby setting the position of the display area 402. The range of the display area 402 may be input as a numerical value by the user using the input unit 170, or may be specified by operating a slide bar that enables specifying of the range of the display area 402 as illustrated in FIG. 5. Further, in a case where the input unit 170 is a touch panel, the user may perform a pinch-in and a pinch-out on the screen, thereby setting the size of the display area 402. Further, the position and the range of the display area 402 may be stored as preset values in, for example, the storage unit included in the computer 150 in the system.

Photoacoustic image data of the display area 402 is thus selectively generated and displayed regardless of the position of the probe 180, whereby it is possible to reduce the calculation load for generating an image. Thus, the present exemplary embodiment is suitable for displaying a moving image.

Further, based on the acoustic waves acquired at the positions Pos1, Pos2, and Pos3, the calculation unit 151 may generate pieces of photoacoustic image data V1 to V3, respectively, in the range of the display area 402 and combine the pieces of photoacoustic image data V1 to V3, thereby calculating combined photoacoustic image data V′1. In the display area 402, the areas irradiated with the light at the respective positions overlap each other. Thus, by combining the pieces of image data V1 to V3, it is possible to obtain the combined photoacoustic image data V′1 in which an artifact is reduced. To further obtain the effect of reducing an artifact by combining, it is desirable that the high-resolution areas should overlap each other in the display area 402.

Further, when combined photoacoustic image data is calculated from pieces of photoacoustic image data, the combining ratios of the pieces of photoacoustic image data V1 to V3 may be weighted.

Further, image values in the plane or the space of the pieces of photoacoustic image data may be weighted. For example, in a case where the area irradiated with the light and the high-resolution area are not the same, an area that is the area irradiated with the light, but is not the high-resolution area can be included in the display area 402. In this case, image values are weighted differently between the high-resolution area and the area other than the high-resolution area, and the pieces of photoacoustic image data are combined, whereby it is possible to obtain combined photoacoustic image data having a high signal-to-noise (S/N) ratio. Specifically, the weighting of a pixel in the high-resolution area is greater than the weighting of a pixel in the area other than the high-resolution area.

Further, although FIG. 4 illustrates the positions of the probe 180 only up to Pos3, photoacoustic waves are also received at positions Pos4, Pos5, . . . , and PosN by continuing the measurements, whereby it is possible to generate pieces of photoacoustic image data V4, V5, . . . , and VN. Furthermore, it is possible to generate pieces of combined photoacoustic image data V′2, V3, . . . , and V′(N−2) from the pieces of photoacoustic image data V4, V5, . . . , and VN. At this time, it is desirable that the position of the probe 180 should move such that the area irradiated with the light or the high-resolution area is included in the display area 402. Further, the probe 180 moves such that the positions PosN do not to overlap each other, whereby it is possible to obtain pieces of combined photoacoustic image data in which an artifact is further reduced. In a case where the probe 180 makes a circling motion as in the present exemplary embodiment, the probe 180 may move such that in at least two successive circling motions, the positions of the probe 180 do not overlap each other at the timings when light is emitted. A “circling motion” in the present exemplary embodiment is a concept including not only a motion along a circular trajectory illustrated in FIG. 4, but also a motion along an elliptical or polygonal trajectory and a linear reciprocating motion.

As a reconfiguration algorithm, used to generate photoacoustic image data, for converting signal data into volume data as a spatial distribution, an analytical reconfiguration method such as a back projection method in a time domain or a back projection method in a Fourier domain, or a model-based method (an iterative calculation method) can be employed. Examples of the back projection method in a time domain include universal back-projection (UBP), filtered back projection (FBP), and phasing addition (delay-and-sum).

Further, the calculation unit 151 may calculate the light fluence distribution, within the subject 100, of light emitted to the subject 100 and divide the initial sound pressure distribution by the light fluence distribution, thereby acquiring absorption coefficient distribution information. In this case, the calculation unit 151 may acquire the absorption coefficient distribution information as photoacoustic image data. The computer 150 can calculate the spatial distribution of light fluence within the subject 100 by a method for numerically solving a transport equation and a diffusion equation representing the behavior of light energy in a medium that absorbs and scatters light.

Furthermore, the processes of steps S320 and S330 may be executed using light of a plurality of wavelengths, and in these processes, the calculation unit 151 may acquire absorption coefficient distribution information corresponding to light of each of the plurality of wavelengths. Then, based on the absorption coefficient distribution information corresponding to light of each of the plurality of wavelengths, the calculation unit 151 may acquire, as photoacoustic image data, spatial distribution information regarding the concentration of a substance forming the subject 100 as spectral information. That is, using signal data corresponding to light of the plurality of wavelengths, the calculation unit 151 may acquire spectral information. As an example of a specific method for emitting light, a method for, every time light is emitted to the subject 100, switching the wavelength of light is possible.

### (Step S340: Process of Displaying (Updating) Photoacoustic Image Data)

As illustrated in FIG. 5, the display unit 160 displays the combined photoacoustic image data corresponding to the display area 402 and created in step S330. At this time, a three-dimensional volume image may be displayed, or a two-dimensional image, such as an MIP from a particular direction or a cross-sectional image based on a particular position, may be displayed. Further, as described in step S330, in a case where a plurality of pieces of combined photoacoustic image data is created, display is updated as needed using an image corresponding to each of the pieces of combined photoacoustic image data, whereby it is possible to continue observing subject information at a certain position in a moving image.

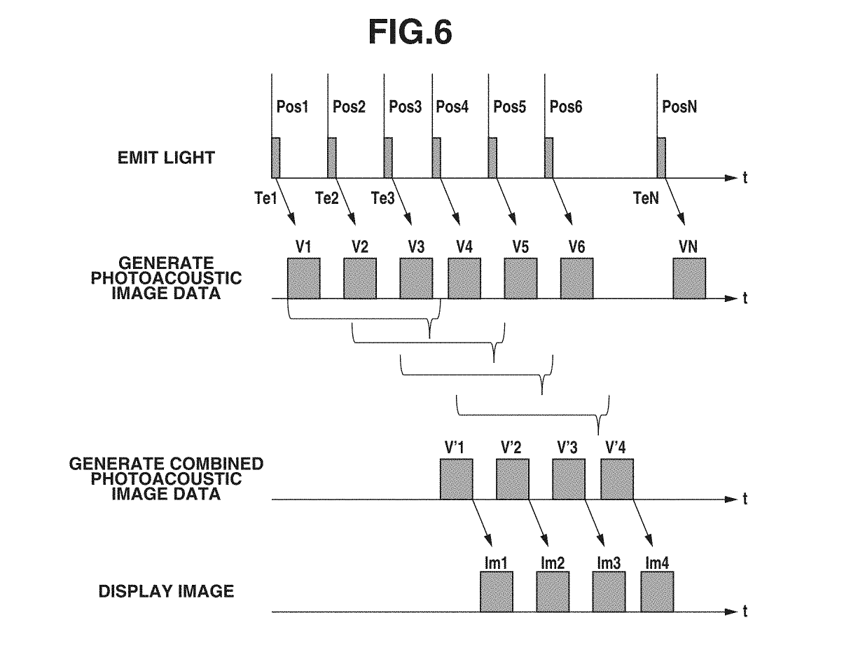

FIG. 6 illustrates a time chart of the flow in FIG. 3. FIG. 6 illustrates the temporal relationships among the timings of light emissions and processes performed by the calculation unit 151 and the display unit 160.

FIG. 6 illustrates timings Te1, Te2, Te3, . . . , and TeN when light is emitted to the subject 100, and the acquisition times of photoacoustic signals acquired according to the respective emissions of light. If photoacoustic signals are acquired by the signal collection unit 140 at the respective timings, the calculation unit 151 generates pieces of photoacoustic image data V1, V2, . . . , and VN by image reconfiguration using the acquired photoacoustic signals. Further, using three pieces of photoacoustic image data, i.e., the pieces of photoacoustic image data V1 to V3, the calculation unit 151 calculates combined photoacoustic image data V′1. In this example, combined photoacoustic image data V′n is generated by combining pieces of photoacoustic image data Vn to V(n+2).

Images Im1, Im2, Im3, . . . based on the thus obtained pieces of combined photoacoustic image data are sequentially displayed at the display unit 160, whereby it is possible to present a photoacoustic image updated in real time. The pieces of photoacoustic image data to be combined are pieces of photoacoustic image data generated by receiving photoacoustic waves at the positions where the areas irradiated with the light or the high-resolution areas overlap each other. Thus, regarding the positions where these overlaps occur, it is possible to generate images in which an artifact is particularly reduced. Further, the display area is the same regardless of the position of the probe 180. Thus, it is possible to continue observing subject information at a certain position in a moving image.

In the present exemplary embodiment, combined photoacoustic image data is generated from a plurality of temporally consecutive pieces of photoacoustic image data. However, not all the consecutive pieces of photoacoustic image data necessarily need to be used. For example, in a case where a light source capable of emitting light at a high repetition frequency, such as a light-emitting diode, is used, and if all the plurality of consecutive pieces of photoacoustic image data is used, the amount of data is enormous. Thus, combined photoacoustic image data is generated by thinning some of the pieces of photoacoustic image data, whereby it is possible to reduce the amount of data in the storage unit and the load of the calculation unit 151.

As illustrated in FIG. 5, the number of pieces of photoacoustic image data used to generate combined photoacoustic image data and the combining ratios of the respective pieces of photoacoustic image data when the combined photoacoustic image data is generated from the pieces of photoacoustic image data may be specified by operating slide bars that enable specifying of the number of pieces of data and the ratios in the display area 402, or may be stored as preset values in, for example, the storage unit included in the computer 150 in the system. Then, for example, based on information regarding a specialty or a part to be observed, the computer 150 may set an appropriate number and appropriate combining ratios from among the preset values. Furthermore, the range (size) of the display area 402 can also be set by the user using a slide bar or the like. This enables the user to easily observe the subject by narrowing down to a range of interest. Further, the user may reference input patient information such as a patient identification (ID), and when capturing the same patient again, display settings similar to the previous settings as candidates, or the computer 150 may automatically set initial values.

According to the present exemplary embodiment, images of an area common to consecutive frames in a plurality of frame images are selectively displayed, thereby reducing flicker in a portion other than the common area. Thus, it is possible to reduce stress that can be felt by an observer when observing a moving image.

With reference to FIG. 4, another exemplary embodiment (a second exemplary embodiment) is described. In the first exemplary embodiment, the areas where the pieces of photoacoustic image data are generated and the display area 402 are equal to each other. In contrast, in the second exemplary embodiment, a case is described where the pieces of photoacoustic image data and the display area 402 have such a relationship that the areas where the pieces of photoacoustic image data are generated are larger than the display area 402 and include the display area 402. Unless otherwise described, an apparatuses, a driving method, and a processing method similar to those of the first exemplary embodiment are also applied to the present exemplary embodiment.

The calculation unit 151 generates pieces of photoacoustic image data V1 to V3 at positions Pos1, Pos2, and Pos3, respectively, such that each of the areas where the pieces of photoacoustic image data are generated is the area irradiated with the light. The calculation unit 151 combines these three pieces of photoacoustic image data V1 to V3 while holding the relative positional relationships among the pieces of photoacoustic image data V1 to V3, thereby generating combined photoacoustic image data V1. The display unit 160 displays as a display image Im1 only an area included in the display area 402 in the combined photoacoustic image data V′1. The processing is also performed on pieces of combined photoacoustic image data V′2, V′3, . . . , and V′(N−2), whereby it is possible to continue observing the subject at a certain position in a moving image similarly to the first exemplary embodiment. That is, the size of the area of each of the pieces of photoacoustic image data generated by the calculation unit 151 corresponding to the respective emissions of light is the same as or greater than that of the high-resolution area, and the display unit 160 displays an image corresponding to only the portion of the display area 402, whereby it is possible to observe the range of the display area 402.

Further, after the pieces of photoacoustic image data V1 to V3 are generated, and when the combined photoacoustic image data V′1 is generated, only areas included in the display area 402 may be combined, thereby obtaining the combined photoacoustic image data V1. Further, after generating the combined photoacoustic image data V1, the calculation unit 151 may hold only an area included in the display area 402 and output the area to the display unit 160.

Not only is a range outside the display area 402 and included in the high-resolution area not displayed at all, but the range can also be displayed by reducing the visibility of the range as compared with the display area 402. Specifically, it is possible to reduce the luminance of the area outside the display area 402 relative to the display area 402, reduce the contrast of the area, increase the transmittance of the area, or perform a masking process on the area. In any of the above cases, in pieces of photoacoustic image data generated by the respective multiple emissions of light, images of an area common to consecutive frames are selectively displayed. This reduces flicker in a portion other than the common area.

Also in the present exemplary embodiment, similar to the first exemplary embodiment, images of an area common to consecutive frames in a plurality of frame images are selectively displayed, thereby reducing flicker in a portion other than the common area. Thus, it is possible to reduce stress that can be felt by an observer when observing a moving image.

### (Others)

The above exemplary embodiments have been described taking as an example a case where a living body is a subject. The disclosure, however, is also applicable to a case where a subject other than a living body is a target.

Further, the exemplary embodiments have been described using a subject information acquisition apparatus as an example. The disclosure, however, can also be regarded as a signal processing apparatus for generating images based on acoustic waves received at a plurality of relative positions to a subject, or a display method for displaying an image. For example, the probe 180, the movement unit 130, and the signal collection unit 140, and the computer 150 can also be configured as different apparatuses, and a digital signal output from the signal collection unit 140 can also be transmitted via a network to the computer 150 at a remote location. In this case, based on the digital signal transmitted from the signal collection unit 140, the computer 150 as a subject information processing apparatus generates an image to be displayed at the display unit 160.

### Other Embodiments

Embodiment(s) of the disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2018-080092, filed Apr. 18, 2018, which is hereby incorporated by reference herein in its entirety.

What is claimed is:

1. A subject information acquisition apparatus comprising:
a light emission unit configured to emit light to a subject;
a probe configured to receive an acoustic wave generated from the subject to which the light is emitted, thereby generating a signal;
an image generation unit configured to generate a plurality of frame images based on signals acquired at a plurality of respective relative positions of the probe to the subject and resulting from acoustic waves from the subject; and
a display control unit configured to selectively display images of an area common to consecutive frames in the plurality of frame images at a display unit.

2. The subject information acquisition apparatus according to claim 1, wherein the image generation unit selectively generates images regarding the area common to the consecutive frames.

3. The subject information acquisition apparatus according to claim 1, wherein the image generation unit generates images based on the signals acquired at the plurality of respective relative positions.

4. The subject information acquisition apparatus according to claim 3, wherein the image generation unit combines at least two images, thereby generating the frame images.

5. The subject information acquisition apparatus according to claim 1, wherein the display control unit displays the frame images as a moving image at the display unit.

6. The subject information acquisition apparatus according to claim 1, further comprising a movement unit configured to move the probe relative to the subject.

7. The subject information acquisition apparatus according to claim 6, wherein, while light is emitted consecutively twice by the light emission unit, the movement unit moves the probe by a distance less than or equal to a size of a high-resolution area determined based on placement of a plurality of acoustic wave detection elements.

8. The subject information acquisition apparatus according to claim 6, wherein the movement unit moves the probe such that the probe makes a circling motion.

9. The subject information acquisition apparatus according to claim 8, wherein the circling motion is a circling motion performed along a circular trajectory.

10. The subject information acquisition apparatus according to claim 8, wherein the movement unit moves the probe such that the probe receives the acoustic waves at the relative positions different from each other in the continuous circling motion.

11. The subject information acquisition apparatus according to claim 1, wherein the light emission unit changes a position to which the light is emitted, in conjunction with the relative positions of the probe relative to the subject.

12. The subject information acquisition apparatus according to claim 11, wherein the light emission unit is formed integrally with the probe.

13. The subject information acquisition apparatus according to claim 1, further comprising an image capturing unit configured to acquire an optical image of the subject.

14. The subject information acquisition apparatus according to claim 13, wherein the display control unit displays the frame images and the optical image at the display unit.

15. The subject information acquisition apparatus according to claim 13, further comprising an input unit configured to input a position on the optical image,
wherein based on the input position, the acoustic waves are received.

16. The subject information acquisition apparatus according to claim 1, wherein the probe includes:
a plurality of acoustic wave detection elements each configured to detect an acoustic wave; and
a supporting member configured to support the plurality of acoustic wave detection elements such that directional axes of the plurality of acoustic wave detection elements concentrate.

17. A subject information processing method comprising:
generating a plurality of frame images based on signals acquired at a plurality of respective relative positions of a probe to a subject and resulting from acoustic waves from the subject to which light is emitted; and
selectively displaying images of an area common to consecutive frames in the plurality of frame images at a display unit.

18. The subject information processing method according to claim 17, wherein images regarding the area common to the consecutive frames are selectively generated.

19. The subject information processing method according to claim 17, wherein images are generated based on the signals acquired at the plurality of respective relative positions.

20. The subject information processing method according to claim 19, wherein at least two images are combined, thereby generating the frame images.

21. The subject information processing method according to claim 17, wherein the frame images are displayed as a moving image at the display unit.

22. The subject information processing method according to claim 17, wherein the frame images and an optical image of the subject are displayed at the display unit.

23. A non-transitory storage medium that stores a program for causing a computer to execute a subject information processing method, the method comprising;
generating a plurality of frame images based on signals acquired at a plurality of respective relative positions of a probe to a subject and resulting from acoustic waves from the subject to which light is emitted; and
selectively displaying images of an area common to consecutive frames in the plurality of frame images at a display unit.
